(19) United States
(12) Patent Application Publication
(10) Pub. No.: US 2024/0010710 A1
(43) Pub. Date: Jan. 11, 2024

(54) METHOD FOR OBTAINING A COMPOSITION COMPRISING HUMAN PLASMA-DERIVED IMMUNOGLOBULIN M

(71) Applicant: GRIFOLS WORLDWIDE OPERATIONS LIMITED, DUBLIN (IE)

(72) Inventors: Myles LINDSAY, Research Triangle Park, NC (US); Thomas P. ZIMMERMAN, Research Triangle Park, NC (US); Michelle WOZNICHAK, Research Triangle Park, NC (US); Deepa SINGH, Research Triangle Park, NC (US); Erin ROMES, Research Triangle Park, NC (US); Natalia ORLOVA, Research Triangle Park, NC (US); Rebecca SILVERSTEIN, Research Triangle Park, NC (US)

(21) Appl. No.: 18/002,245

(22) PCT Filed: Jul. 8, 2021
(86) PCT No.: PCT/EP2021/069000

§ 371 Date: Dec. 16, 2022

Related U.S. Application Data
(60) Provisional application No. 63/050,611, filed on Jul. 10, 2020.

(51) Int. Cl. C07K 16/06; A61K 39/395; A61K 47/18; A61K 47/26

(52) U.S. Cl. CPC C07K 16/065; A61K 39/39591; A61K 47/183; A61K 47/26

(57) ABSTRACT

A method for preparing a composition of human plasma-derived immunoglobulin M (IgM) includes PEG precipitation of the IgM, resuspension of the precipitated IgM; (c) performing an adsorption chromatography, removing isoagglutinins A/B, nanofiltration, and ultrafiltration/diafiltration. The precipitation can be performed at a pH between 4.5 and 6.5, and the PEG can be at a concentration between 5 (w/v) and 11% (w/v).

### BACKGROUND

### Field

The present disclosure is related to the field of pharmaceutical products. Certain embodiments herein relate to methods for obtaining a composition comprising immunoglobulin M (IgM), which can be used for many therapeutic indications.

### Description of the Related Art

As normal human plasma contains a substantial amount of IgM, it may be practical and economically viable to harness therapeutic potential of these IgM through the generation of therapeutic preparations. Indeed, an IgM-enriched immunoglobulin preparation, Pentaglobin, of which 12% of the total immunoglobulin content is reported to be IgM, has been successfully used for treating infections associated with sepsis in patients, as well as transplant rejection, and for certain inflammatory conditions in experimental models. Such preparations may also provide benefits to combat infections that arise in patients with autoimmune disease.

A plasma-derived polyclonal IgM pharmaceutical composition suitable for human administration can be used to treat systemic antibiotic resistant bacterial infections (bacteremia), an area of unmet clinical need, though other indications may be considered. IgM circulates in plasma primarily in its pentameric form, comprised of 5 identical IgM monomer subunits connected by disulfide bonds.

IgM pharmaceutical compositions are not prevalent most likely due to the difficulty associated with production of pure IgM solutions at concentrations suitable for therapeutic use. Moreover, its purification is complicated by the size of the protein (>6 times the molecular weight of IgG) and its tendency to self-associate into higher molecular weight species that may be inactive or potentially pose immunogenic or other risk to patients. The polyclonal nature of these pharmaceutical compositions provides an even greater challenge due to the lack of homogeneity of the antibodies, wherein different levels of solubility may be associated with different IgM populations.

In addition, since IgM is the most associated antibody with blood type mismatch agglutination/hemolysis, levels of IgM that bind to blood group A and B antigens on the surface of red blood cells (RBCs) must be reduced.

The characteristics desired in IgM pharmaceutical compositions include high purity (IgM content >97%), high activity as measured both by specific binding affinity to clinically relevant bacterial antigens and capacity to activate complement, reduced isoagglutinin titers, minimal capacity to activate complement non-specifically, and <10% aggregated species, defined in this context to include both reversible and irreversible species of a size greater than pentamer.

In view of the above, there is still the need to provide a process for obtaining human plasma-derived IgM that overcome said drawbacks. The present inventors have developed a process for obtaining IgM pharmaceutical compositions to overcome the challenges typically associated with this protein. Throughout the process, steps are taken to minimize levels of IgM aggregates. This is done by understanding conditions under which IgM is prone to self-association, many of which are encountered during the course of purification. These conditions include high IgM concentration, exposure to a pH near its isoelectric point (range for polyclonal human IgM of 5.5-7.4), certain combinations of high/low ionic strength and neutral/acidic pH and mechanical stresses. In addition, a stabilizer, arginine, is added to certain steps of the process to inhibit IgM self-associations and to dissociate reversibly self-associated aggregates. To address isoagglutinin titers, this product also incorporates affinity chromatography specific for those IgM that bind to A/B RBC surface antigens to enhance safety.

Ultimately, the process allows for a safe, high purity, high concentration polyclonal IgM product. For comparison, the only product now commercially available, Pentaglobin, which claims to be an enriched IgM therapy, is comprised of only 12% IgM, with the remaining 88% being IgG and IgA. That product has a reported IgM concentration of around 6 g/L. The composition obtained by the present invention will be at least 97% IgM as assessed by immunonephelometry, have an aggregate content of <10% and an IgM concentration of ≥15 g/L, with a product of 50 g/L or greater being feasible by the described process.

The process of the present invention comprises steps to purify and concentrate polyclonal IgM from human plasma. Efforts were made to ensure logical flow of the unit operations, requiring minimal manual intervention (pH adjustments, concentration/dilution, ionic strength adjustments etc.) between steps. Unit operations dedicated to buffer exchange, impurity reduction, isoagglutinin reduction, pathogen clearance capacity and formulation were developed and implemented. These operations were designed such that formation of IgM aggregates is minimized. The process also includes steps wherein those aggregates that are present are removed or converted back to mono-pentamer.

### BRIEF DESCRIPTION OF THE DRAWINGS

FIG. 1 shows the comparison of SEC-HPLC profiles before (ANX strip) and after PEG precipitation (PEG Suspension). Note the reduction of high MW IgM species. Some reduction in the abundance of IgG and IgA is also observed. Regions of the chromatogram were identified by MALS analysis estimation of molecular weights. Pentamer ˜930 kDa while di-pentamer is ˜1.8 MDa. The higher MW aggregate region is relatively polydisperse with molecular weight greater than di-pentamer.

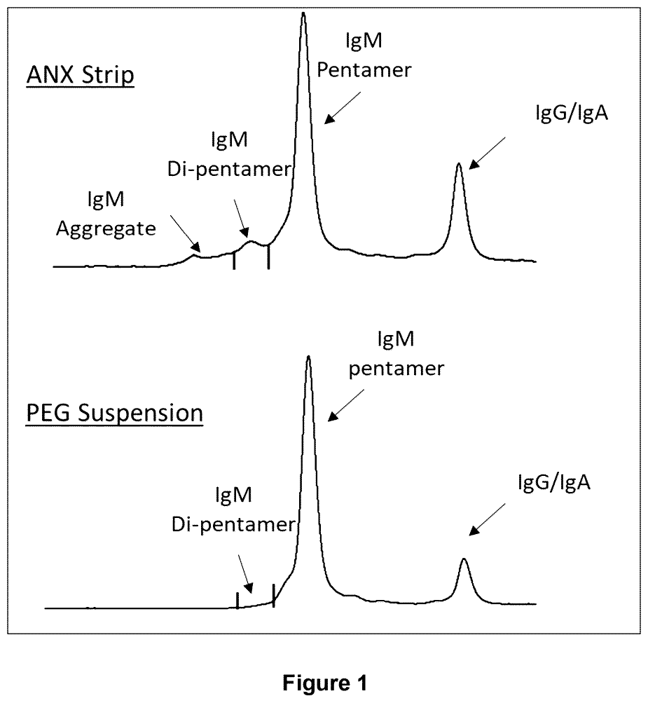

FIG. 2 shows the effect of pH on concentration of IgM prior to diafiltration. a) shows the pre-concentration SEC-HPLC profiles (2 mg/mL); and b) post-concentration SEC-HPLC profiles (20 mg/mL).

FIG. 3 shows the effect of UF/DF load pH on post-formulation IgM pentamer content of IgM formulated to 25 mg/mL.

FIG. 4 shows the reduced SDS-PAGE of purified IgM composition. Starting material (ANX Strip) compared to final product (formulated Bulk). Band identification indicated in the figure.

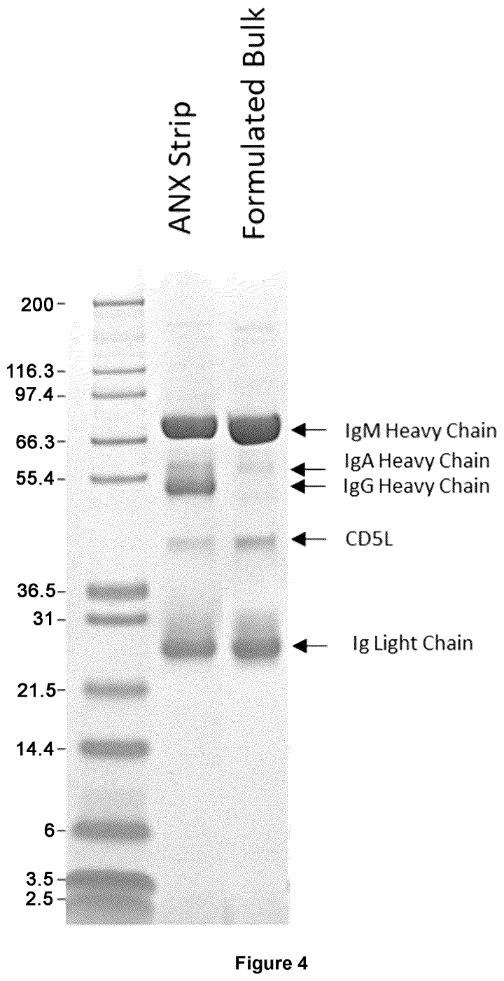

FIG. 5 shows a diagram related to the process to purify IgM from pooled human plasma.

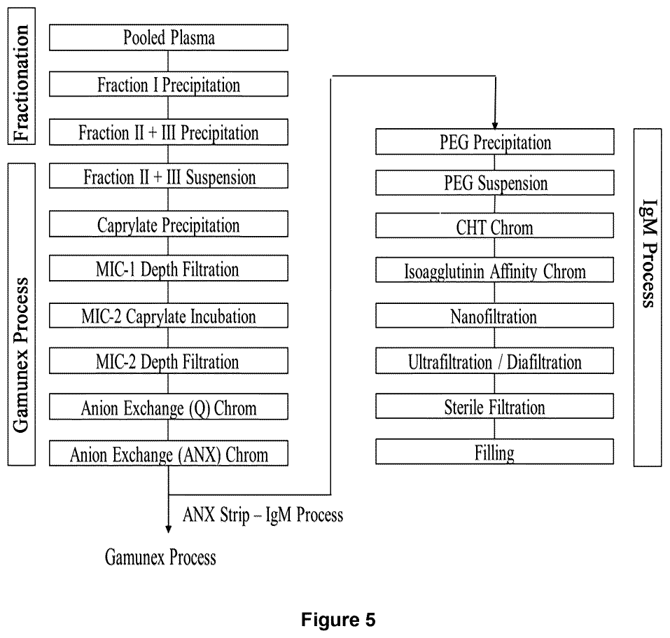

### SUMMARY

In a first aspect, the present invention refers to a method for preparing a composition of human plasma-derived immunoglobulin M (IgM) comprising the steps of:
a) precipitation of said IgM using polyethylene glycol (PEG);
b) resuspension of the precipitated IgM;
c) adsorption chromatography;
d) removing isoagglutinins A/B;
e) nanofiltration; and
f) ultrafiltration/diafiltration.

In one embodiment, said precipitation step a) is performed at a pH between 4.5 and 6.5.

In one embodiment, said PEG is at a concentration between 5% (w/v) and 11% (w/v). Preferably, said PEG is PEG-3350.

In one embodiment, said absorption chromatography is ceramic hydroxyapatite (CHT) chromatography.

In one embodiment, the loading solution of the ceramic hydroxyapatite CHT comprises NaCl, preferably at a concentration between 0.5 M and 2.0 M.

In one embodiment, the washing solution of the ceramic hydroxyapatite CHT comprises urea, preferably at a concentration between 1 M and 4 M.

In one embodiment, said step d) of removing isoagglutinins A/B is performed by affinity chromatography using A/B oligosaccharides as ligand.

In one embodiment, said step d) of removing isoagglutinins A/B is performed using at least two affinity columns in series, at least one with oligosaccharide A as a ligand, and at least one with oligosaccharide B as a ligand or step d) is performed using at least one affinity column containing a mixture with oligosaccharide A and oligosaccharide B as a ligand.

In one embodiment, said nanofiltration step e) is performed through a filter of 35 nm or greater of average pore size.

In one embodiment, said nanofiltration step e) is performed using a buffer comprising at least 0.5 M of Arginine-HCl at a pH between 6.0 and 9.0. Preferably, said nanofiltration step e) is performed using a buffer comprising at least 0.5 M of Arginine-HCl at a pH between 7.0 and 8.0.

In one embodiment, said initial ultrafiltration concentration step is performed at a pH between 4.5 and 5.0 and in the presence of surfactant. In one embodiment, said surfactant is polysorbate 80 (PS80) or polysorbate 20 (PS20).

In one embodiment, said diafiltration step e) is performed with a succinate buffer or containing amino acids at a pH between 3.8 and 4.8.

In one embodiment, said amino acids are glycine, alanine, proline, valine, or hydroxyproline or a mixture thereof.

In another aspect, the present invention discloses a storage stable, liquid composition comprising:
i) about 1.5% to about 5% w/v polyclonal IgM, the polyclonal IgM being at least 90% by weight of the total protein content of the composition;
ii) an amino acid selected from the group consisting of glycine, alanine, proline, valine, or hydroxyproline, and combinations thereof, at a concentration of about 0.15 M to about 0.45 M; and
iii) a pH of about 3.8 to about 4.8,
iv) a surfactant selected between polysorbate 80 (PS80) and polysorbate 20 (PS20),
   wherein, the composition is substantially depleted of isoagglutinin A and isoagglutinin B; and the composition is stable in liquid form for at least 24 months when stored at 2 to 5° C., such that the content of IgM aggregates having a molecular weight ≥1200 kDa in the composition remains less than or equal to 10 by weight of the total protein (immunoglobulin) content of the composition, as determined by high performance size exclusion chromatography.

In one embodiment, the concentration of said surfactant is greater than 20 ppm.

In one embodiment, the concentration of said IgM is from about 2.0% to about 3.0% w/v.

In one embodiment, said composition, further comprises IgG at a concentration of less than about 0.1% w/v.

In one embodiment, said composition further comprises IgG, wherein the IgG is less than 1% by weight of the total protein concentration.

In one embodiment, said composition further comprises IgA at a concentration of less than about 0.15% w/v.

In one embodiment, said composition further comprises IgA, wherein the IgA is less than 3% by weight of the total protein concentration.

In one embodiment, said amino acid is glycine.

In one embodiment, the concentration of the glycine is about 0.2 M to about 0.3 M.

In one embodiment, said composition is stable for at least 24 months.

In one embodiment, the polyclonal IgM is human plasma-derived IgM.

In one embodiment, the pH is from 4.0 to 4.4.

In one embodiment, the IgM aggregates remain less than or equal to 10% by weight of the total protein content of the composition.

### DETAILED DESCRIPTION

In the process of the present invention, the starting material used can come from different sources. For example, the source material for the described IgM process can be column strip from either of the two Gamunex process (as described in U.S. Pat. 6,307,028) anion-exchange chromatography columns (Q sepharose or ANX sepharose) operated in series. In that process, IgG is purified from Fraction II+III paste generated from the Grifols plasma fractionation processes, as described in the mentioned patent. Briefly, after collecting IgG in the anion exchange columns flow through, bound protein, almost exclusively immunoglobulin (IgM, IgG and IgA), is eluted by applying a buffer comprising 0.5 M sodium acetate at pH 5.2. Columns are stripped separately wherein either or both fractions can be further processed to purify IgM. The abundance ratios of each of the three immunoglobulins differ significantly between the two column strips.

Due to the buffer environment in which the Gamunex column anion exchange strips are collected (high acetate), buffer exchange is desired prior to subsequent ceramic hydroxyapatite (CHT) chromatography. The CHT column is not compatible with high concentrations of acetate, which are known to degrade the performance of the resin over time. In addition, because the anion exchange columns were not optimized for IgM purification, IgM in the column strips tend to be moderately self-associated, often containing >10% high MW IgM species. To achieve a rapid and efficient buffer exchange and to improve the IgM pentamer composition, IgM is precipitated at slightly acidic pH (5-6) by addition to 7.0% to 11% (target 10%) (w/w) polyethylene glycol (PEG)-3350. IgM is fully precipitated in less than 1 hour. Precipitated IgM is recovered by depth filtration in the presence of 0.5% filter aid or by centrifugation. Collected precipitate can be recovered and stored frozen or processed immediately. Typically, IgM collected by depth filtration is then rapidly resolubilized by recirculating a buffer solution compatible with CHT column operation and maximal IgM solubility through the depth filter for ≤30 minutes. The volume of buffer used (typically half the volume of the starting material) is selected to minimize the volume of CHT column load while also not resulting in over concentration of IgM. This buffer comprises 5 mM sodium phosphate, 20 mM tris, 1 M NaCl pH 8.0. Using PEG precipitation for buffer exchange instead of the more common UF/DF allows for gentle treatment of the protein, since pumping and mixing are minimized, as well as a rapid shift through a pH environment in which IgM aggregation is most prominent (pl range of polyclonal IgM: pH 5.5-7.4). Resulting IgM is almost exclusively in the mono-pentameric form, with no larger IgM species detected (see Table 1). Some limited purification of IgM, primarily by reduction in IgG which remains partially soluble under these precipitation conditions, also occurs across this step. Removal of aggregated immunoglobulin species by this PEG precipitation/solubilization method has not been reported in literature according to a cursory search.

Table 1 shows the IgM profile both pre and post precipitation by PEG and resolubilization. Values in parenthesis are calculated percentages of the different IgM species compared to overall IgM content and do not include species with MW<IgM pentamer, predominantly IgG and IgA. Data represent averages from four clinical scale process runs. Aggregate, di-pentamer and pentamer are identified by MALS analysis.

TABLE 1
IgM profile both pre and post precipitation by PEG and resolubilization.

| | SEC-HPLC | | | | | |
|---|---|---|---|---|---|---|
| | IgM | IgM Di- | IgM | MW< | Acetate | IgM Purity |
| | aggregate | pentamer | pentamer | pentamer | concentration | (% total |
| | (%) | (%) | (%) | (%) | (mM) | immunoglobulin) |
| AEX | 3.6% | 6.3% | 61.5% | 24.9% | 179 | 72.0% |
| Column | (9.6%) | (8.4%) | (82.1%) | | | |
| Eluate | | | | | | |
| PEG | 0% | 0.8% | 85.7% | 13.5% | 10 | 84% |
| Suspension | (0%) | (0.9%) | (99.1%) | | | |

The primary step to affect separation of IgM from impurities is ceramic hydroxyapatite chromatography. Polyclonal plasma-derived IgM was found to have a high affinity for this resin, with all present isoforms putatively binding through the Ca²⁺ mechanism. To allow for maximum binding capacity and IgM solubility as well as to simplify operation, IgM is loaded in a high salt environment (1 M NaCl). In this solution, IgG largely does not bind the resin since the nature of its interaction with hydroxyapatite appears to be ionic. IgA also appears to largely bind under this condition. Because IgM and IgA elute from the resin at similar phosphate concentrations, it was not feasible to separate the two proteins using a phosphate buffer gradient or isocratic elution. To displace IgA and residual IgG the column is washed with a solution containing 5 mM sodium phosphate, 1 M NaCl, 2 M urea at pH 8.0. The mechanism by which this purification is affected is not known, though it is thought to be a result of the perturbation of the IgA Ca²⁺ binding moiety due to partial denaturation by urea or due to the dissociation of non-covalent complexes of IgM and IgA. However, IgM appears to be resistant to elution by urea as it remains completely bound to the resin under these conditions. Higher concentrations of urea (up to 4 M) were tested, with IgM still maintaining significant binding to the resin. Since only minor purification improvement is achieved, however, the additional IgM yield loss at urea concentrations >2 M was not deemed sufficient to justify its use. Once washed, the column is then eluted isocratically with 0.25 M sodium phosphate pH 8.0. Despite significant concentration to >5 g/L, IgM remains essentially free of aggregates as shown in Table 5.

IgM is the antibody most responsible for red blood cell (RBC) hemolysis due to blood type mismatch. Because plasma pools are not segregated by donor blood type, those IgM antibodies that bind blood group A/B antigens need to be reduced in abundance. Isoagglutinin titers in the IgM composition of the present invention are reduced by application of the product to resins in which A/B oligosaccharides are immobilized onto the surface. The method of the present invention has already been successfully applied to IgG products, but has not been reported for polyclonal plasma-derived IgM. Columns packed with either anti-A or B resins are run in series, wherein the process stream is applied to the first column and the flow through from the first column is applied directly to the second. The columns are run under conditions where isoagglutinin binding is optimal, which includes ensuring that aggregates of IgM, wherein binding sites could be masked, are minimized. These conditions include applying sample at low concentration (<10 mg/mL) at between about 2-25° C., and at slightly basic pH (8-9). As an example, anti-A titer as measured by flow cytometry is reduced by 4-6 fold through this method (Table 2). Note that these two resins may be blended and packed into a single column with similar results.

Table 2 shows isoagglutinin A titer reduction across the isoagglutinin affinity columns from four runs. Titer as measured by IgM specific flow cytometry.

TABLE 2
isoagglutinin A titer reduction across
the isoagglutinin affinity columns.

| | Load Titer | FT Titer | Fold |
|---|---|---|---|
| | (anti-A) | (anti-A) | Reduction |
| Run 1 | 1057 | 176 | 6.0 |
| Run 2 | 961 | 206 | 4.7 |
| Run 3 | 856 | 248 | 3.5 |
| Run 4 | 937 | 237 | 4.0 |

Due to its large size, IgM has proven difficult to nanofilter. A single IgM pentamer is larger than many viruses and is not amenable to filtration by small pore nanofilters. Larger pore devices, (35 nm and above) have also proven problematic as multimers of IgM, even if they are weakly associated and reversible, will rapidly foul the filter and seize flow. This prevents nanofiltration at IgM concentrations typically encountered during processing (>0.5 mg/mL). To address this issue, the buffer environment of the nanofilter load has to be changed. Agents that prevent protein interactions can be used to aid nanofiltration of large molecules, and for IgM, that proved successful. Arginine-HCl at high concentrations (≥1 M) and near neutral pH (7-8) was effective at increasing the capacity of an Asahi Kasei Planova 35N nanofilter to >400 g IgM per m² of nanofilter area and improved flux significantly at IgM concentrations up to 2 g/L. At lower arginine concentrations (<0.5 M) or lower pH (4.4), the same improvement in filtration properties was not observed.

An additional benefit of the addition of arginine is that it provides extra assurance of process robustness with respect to content of high MW forms of IgM. Arginine at 1 M at pH 6-9 is sufficient to dissociate most reversible IgM aggregates generated during normal processing, thus stabilizing and preparing the composition for final formulation. IgM is a challenging molecule to stabilize and is known for its propensity to self-associate, especially when purified at high concentration or when subjected to mechanical stress. All of these conditions are prevalent during final UF/DF and formulation, wherein the purified product is exposed to vigorous pump cycling/mixing for several hours and where it is concentrated to its formulation target (≥20 mg/mL). To achieve a product devoid of aggregated IgM species, a four step approach to formulation was developed. Given the target formulation comprises IgM at ≥20 mg/mL in a succinate buffer containing an amino acid (glycine/alanine) at pH 3.8-4.8, the protein environment changes significantly from the pH 7-8 and high phosphate/arginine/chloride buffer of the nanofiltrate. In addition, IgM concentration is increased anywhere from 15 to 40 fold.

To achieve the desired IgM formulation, adjustment of the composition pH through the isoelectric point of the protein (5.5-7.4 for polyclonal plasma-derived IgM), wherein aggregate formation is most prominent, is necessary. One approach to pH adjustment is to allow the pH of the material to shift gradually during diafiltration against the low pH formulation buffer. This approach has proven problematic for IgM in that the protein solubility diminishes greatly in the relatively broad IgM pl range, resulting in on-system precipitation of the product and subsequent fouling of the ultrafiltration membrane. As this gradual pH shift occurs, concentrations of arginine useful for inhibition of IgM self-associations are no longer present due to simultaneous buffer exchange. Rapidly adjusting the pH of the product by acid addition (1 N HCl, 1 M acetic acid or 0.5 M succinic acid) through the pl (<5.0) in the presence of 1 M arginine was found to completely prevent precipitation.

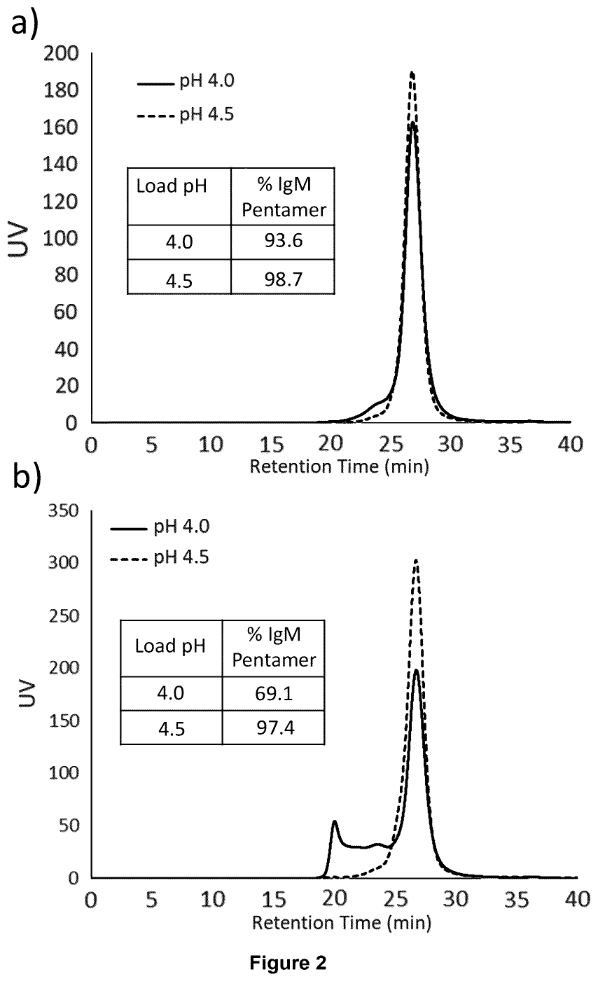

The second step in IgM formulation is to concentrate (UF1) the protein to greater than 20 mg/mL in order to optimize buffer usage during diafiltration. This is a challenging step in that it is the first time that IgM will experience a concentration wherein aggregation becomes especially problematic and rapid. Due to its large size and resultant slow rate of diffusion, local concentrations of IgM on the surface of the TFF membrane are anticipated to be even higher. As such, it is important that IgM be in an environment amenable to stability of the pentamer. Despite the final formulation target pH of 3.8-4.8 and the observation that IgM is less prone to self-association in this pH range, it was surprisingly found that the optimal pH for concentration is higher, in the range of 4.5-5.0. The dramatic difference in high MW IgM content when concentrated at pH 4.0 compared to 4.5 is shown in FIG. 2. The reason for this observation is unclear, though it seems likely that the effectiveness of arginine at inhibiting IgM self-interactions is significantly diminished at pH below 4.4. This hypothesis is supported by the lack of success of arginine as a low pH formulation excipient for IgM, as well as the failure of 1 M arginine to improve nanofilterability at low pH. When concentrated at a pH below 4.4, preferably below 4.2, self-associated IgM species were generated ranging from di-pentamer to large aggregates, most of which remain in the final formulated product even after diafiltration.

After concentration at pH ≥4.5, the solution must be buffer exchanged. To accomplish this, the concentrated IgM solution at pH 4.5-5.0 is diafiltered against a succinate buffer (≥5 mM) wherein phosphate and arginine are removed and the pH is simultaneously shifted to the final formulation target (3.8-4.8). This diafiltration buffer may also contain an amino acid(s) (glycine/alanine/proline/valine/hydroxylproline), also part of the final IgM formulation. Importantly, diafiltration appears to result in limited dissociation of even the most highly aggregated species generated by low pH (<4.4) concentration. At least some IgM aggregates do not appear to be reversible, however, as upon mild to moderate heating at 37° C. (known to dissociate reversibly self-associated IgM species; data not shown), dilution, or by addition of arginine, complete recovery of the IgM mono-pentamer cannot be achieved. As a result, the initial concentration step must be performed at a pH >4.4, but most preferably ≥4.5.

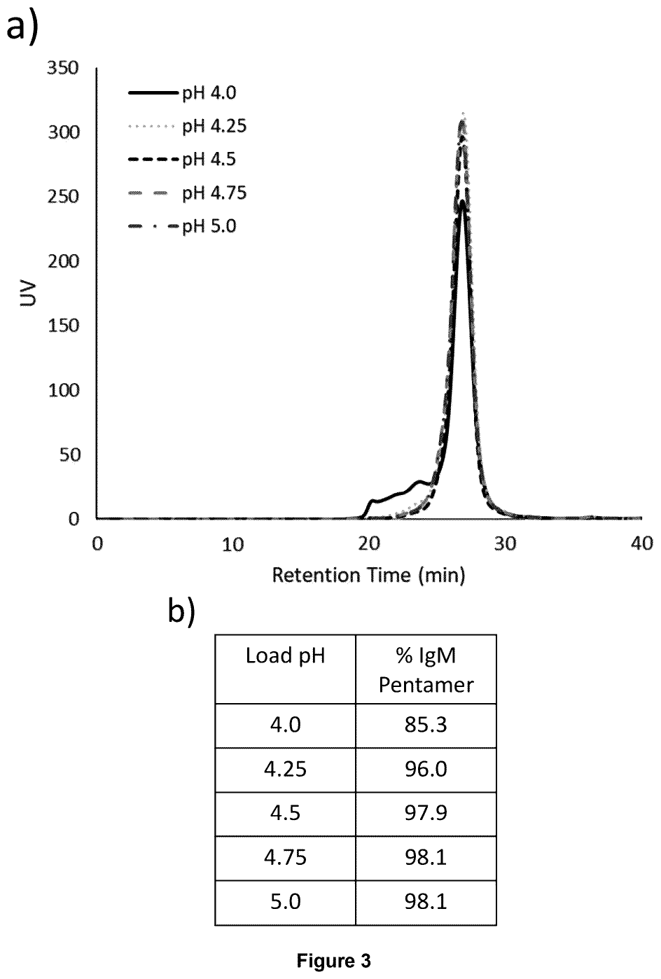

Once exchanged into succinate buffer, IgM can be further concentrated to its formulation target. For 25 mg/mL formulation, for example, the final concentration can be in the range of 30-35 mg/mL to allow for a system rinse to be added back to the product to improve recovery. For a higher concentration product, 50 mg/mL IgM for example, it has been shown feasible to concentrate up to 80 g/L without generating significant quantities of aggregated IgM. These high concentrations also allow for addition of excipients. Levels of IgM aggregation in final formulated 25 mg/mL IgM products, generated at UF/DF load pH ranging from 4.0 to 5.0, are shown in FIG. 3.

In addition to the IgM aggregates that form as a consequence of concentration in a low pH environment, IgM has been shown to form much larger aggregates as a result of certain types of physical stress including pumping/mixing and exposure to air/liquid interfaces. This is especially prominent at high concentration. To prevent formation of these large aggregates, surfactant, namely polysorbate 20 or 80, can be added to the IgM solution prior to UF/DF. Addition of polysorbate dramatically improves the step yield. The mechanism for this improvement is not fully understood at this time, but may be a result of reduced IgM adsorption to process surfaces or by preventing formation of large aggregates at air/liquid interfaces that could subsequently accumulate on the filter surface. Addition of surfactant also improves the formulated bulk appearance and filterability.

The ultimate effect of this overall process is the generation of a very pure (>97% total immunoglobulin), high concentration IgM liquid product with >98% pentamer content and high visual clarity as shown in Table 3.

Table 3 shows IgM final product characteristics. Results are from four formulations of 25 mg/mL and one formulation of 50 mg/mL.

TABLE 3
IgM final product characteristics.

| | | | | SEC-HPLC | | | |
|---|---|---|---|---|---|---|---|
| | | IgM Purity | | IgM | IgM di- | IgM | MW< |
| | IgM | (% total | | aggregate | pentamer | pentamer | pentamer |
| Batch | (g/L) | Immunoglobulin) | NTU | (%) | (%) | (%) | (%) |
| Run 1 | 25 | 98.5% | 9.33 | 0.2% | 0.6% | 98.8% | 0.2% |
| Run 2 | 25 | 97.8% | 8.92 | 0.2% | 0.5% | 98.8% | 0.5% |
| Run 3 | 25 | 97.9% | 7.83 | 0.1% | 0.2% | 99.3% | 0.3% |
| Run 4 | 25 | 98.5% | 9.9 | 0.2% | 0.9% | 98.6% | 0.1% |
| Run 5 | 50 | 98.1% | — | 0.5% | 1.7% | 97.4% | 0.2% |

In addition, IgM purified via this process robustly maintains binding affinity for multiple relevant bacterial antigens as well as the ability to induce specific complement activation as measured by our potency assay as shown in Table 4.

Activity and binding characteristics of starting material (ANX Strip) and formulated bulks from the IgM process. Values represent the average of four runs with standard deviations in parenthesis. Values per mL normalized to IgM content measured by nephelometry (mg/mL).

TABLE 4
IgM ability to induce specific complement
activation as measured by potency assay.

| | | Antigen Binding | | |
|---|---|---|---|---|
| | | | | P. |
| | | E. coli | K. | aeroginosa |
| | Potency | LPS | pneumoniae | Flagelin |
| Material | (U/mg) | (U/mg) | LPS (U/mg) | (U/mg) |
| ANX | 2.78 | 2.80 | 3.69 | 2.72 |
| Strip | (0.23) | (0.36) | (0.95) | (0.64) |
| Form. | 3.16 | 3.12 | 3.62 | 3.34 |
| Bulk | (0.14) | (0.28) | (0.07) | (0.49) |

The effectiveness of the described process to eliminate and prevent formation of IgM aggregates is depicted in Table 5. After PEG precipitation and resuspension, levels of IgM aggregate remain minimal throughout the process, even when concentrated to >20 g/L.

SEC-HPLC analysis of IgM process fractions from IgM purification was performed. Values represent averages from four runs with standard deviations shown in parenthesis. MW<pentamer % for samples upstream of CHT eluate were not included due to high IgG/IgA content.

TABLE 5
IgM aggregates at the end of the process.

| | SEC-HPLC | | | |
|---|---|---|---|---|
| | IgM Aggregate | IgM Di- | IgM Pentamer | IgM MW< |
| Process Fraction | (%) | pentamer (%) | (%) | Pentamer (%) |
| ANX Strip | 9.6% (5.1%) | 8.4% (1.0%) | 82.1% (5.9%) | — |
| PEG Suspension | ND | 0.9% (0.4%) | 99.1% (0.4%) | — |
| CHT Eluate | ND | 1.9% (0.2%) | 97.9% (0.4%) | 0.2% (0.1%) |
| Iso FT | ND | 2.2% (0.2%) | 97.7% (0.2%) | 0.1% |
| | | | | (0.1%)bacust |
| Nanofiltrate | ND | 1.4% (0.2%) | 98.4% (0.3%) | 0.2% (0.2%) |
| (+1M Arginine) | | | | |
| Formulated Bulk | 0.2% (0.1%) | 0.6% (0.3%) | 98.9% (0.3%) | 0.3% (0.2%) |

ND = not detectible.

The formulated bulk was sterile filtered and aseptically filled into glass vials, and stored as a liquid. The composition is stable in liquid form for at least 24 months when stored at 2 to 5° C., such that the content of IgM aggregates having a molecular weight ≥1200 kDa in the composition remains less than or equal to 10% by weight of the total protein (immunoglobulin) content of the composition, as determined by high performance size exclusion chromatography, as shown in Table 6.

TABLE 6
IgM aggregates at the end of 24 months
stored in glass vials at 2 to 5° C.

| | | SEC-HPLC | | | |
|---|---|---|---|---|---|
| | | IgM | IgM di- | IgM | MW< |
| | | aggregate | pentamer | pentamer | pentamer |
| Batch | IgM (g/L) | (%) | (%) | (%) | (%) |
| Run 1 | 25 | 3.2% | 4.7% | 90.9% | 1.2% |
| Run 2 | 25 | 3.1% | 4.8% | 90.8% | 1.2% |
| Run 3 | 25 | 3.9% | 4.8% | 90.0% | 1.3% |
| Run 4 | 25 | 3.0% | 3.5% | 92.5% | 1.0% |
| Run 5 | 50 | 8.2% | 5.4% | 85.3% | 1.0% |

The process used to prepare the IgM of the present invention comprises two steps with capacity to clear/inactivate enveloped viruses and one step to clear non-enveloped viruses. Precipitation by caprylate (19-25 mM) and subsequent depth filtration at low temperature (0-5° C.) and a pH (3.8-4.4) has demonstrated significant capacity to clear non-enveloped viruses. Exposure to 18-26 mM caprylate at higher temperatures (24-27° C.) ) and pH (5.0-5.2) has been demonstrated to be sufficient to inactivate enveloped viruses. Under these conditions, IgM activity does not appear to be compromised. Additional enveloped virus removal has been demonstrated across the 35N nanofilter present in the IgM process.

### Definitions

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this detailed description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

1. A method for preparing a composition of human plasma-derived immunoglobulin M (IgM) comprising:
a) PEG precipitation of said IgM;
b) resuspension of the precipitated IgM;
c) performing an adsorption chromatography;
d) removing isoagglutinins A/B;
e) nanofiltration; and
f) ultrafiltration/diafiltration.

2. The method for preparing the composition according to claim 1, wherein said precipitation step-a) is performed at a pH between 4.5 and 6.5.

3. The method for preparing the composition according to claim 1, wherein said PEG is at a concentration between 5 (w/v) and 11% (w/v).

4. The method for preparing the composition according to claim 1, wherein said absorption chromatography is ceramic hydroxyapatite (CHT) chromatography.

5. The method for preparing the composition according to claim 4, wherein the loading solution of the ceramic hydroxyapatite (CHT) chromatography comprises 0.5 to 2.0M of NaCl.

6. The method for preparing the composition according to claim 4, wherein the washing solution of the ceramic hydroxyapatite (CHT) chromatography comprises urea at a concentration between 1 M and 4 M.

7. The method for preparing the composition according to claim 1, wherein said step d) of removing isoagglutinins A/B is performed by affinity chromatography using A/B oligosaccharides as ligand.

8. The method for preparing the composition according to claim 1, wherein said d) of removing isoagglutinins A/B is performed using at least two affinity columns in series, at least one with oligosaccharide A as a ligand, and at least one with oligosaccharide B as a ligand or said d) is performed using at least one affinity column containing a mixture with oligosaccharide A and oligosaccharide B as a ligand.

9. The method for preparing the composition according to claim 1, wherein said nanofiltration e) is performed through a filter of 35 nm or greater of average pore size.

10. The The method for preparing the composition according to claim 1, wherein said nanofiltration e) is performed using a buffer comprising at least 0.5 M of Arginine-HCl at a pH between 6.0 and 9.0.

11. The method for preparing the composition according to claim 1, wherein said initial ultrafiltration concentration is performed at a pH between 4.5 and 5.0 and in the presence of polysorbate 80.

12. The method for preparing the composition according to claim 1, wherein said diafiltration step is performed with a succinate buffer or containing amino acids at a pH of about 3.8 to about 4.8.

13. The method for preparing the composition according to claim 12, wherein said amino acids are glycine, alanine, proline, valine, or hydroxyproline, or a mixture thereof.

14. A storage stable, liquid composition comprising:
i) about 1.5% to about 5% w/v polyclonal IgM, the polyclonal IgM being at least 90% by weight of the total protein content of the composition;
ii) an amino acid selected from the group consisting of glycine, alanine, proline, valine, or hydroxyproline, and combinations thereof, at a concentration of about 0.15 M to about 0.45 M;
iii) a pH of about 3.8 to about 4.8; and
iv) a surfactant selected between polysorbate 80 (PS80) and polysorbate 20 (PS20),
wherein the composition is substantially depleted of isoagglutinin A and isoagglutinin B; and the composition is stable in liquid form for at least 24 months when stored at 2 to 5° C., such that the content of IgM aggregates having a molecular weight ≥1200 kDa in the composition remains less than or equal to 10% by weight of the total protein (immunoglobulin) content of the composition, as determined by high performance size exclusion chromatography.

15. The composition according to claim 14, wherein the IgM is from about 2% to about 3% w/v.

16. The composition, according to claim 14, further comprising IgG at a concentration of less than about 0.1% w/v.

17. The composition according to claim 14, further comprising IgG, wherein the IgG is less than 1% by weight of the total protein concentration.

18. The composition according to claim 14, further comprising IgA at a concentration of less than about 0.15% w/v.

19. The composition according to claim 14, further comprising IgA, wherein the IgA is less than 3% by weight of the total protein concentration.

20. The composition according to claim 14, wherein the amino acid is glycine.

21-25. (canceled)
